(19) United States
(12) Patent Application Publication
(10) Pub. No.: US 2005/0228426 A1
(43) Pub. Date: Oct. 13, 2005

(54) Surgical device

(76) Inventor: Alexander Craig Wilhem Campbell, Glasgow (GB)

Correspondence Address: Tarolli Sundheim Covell & Tummino, 526 Superior Avenue, Suite 1111, Cleveland, OH, 44114-1400, US

(21) Appl. No.: 10/505,919

(22) PCT Filed: Dec. 10, 2002
(86) PCT No.: PCT/GB02/05582

§ 371 Date: Apr. 19, 2005

(30) Foreign Application Priority Data
Feb. 28, 2002 (GB) 0204850.2

(51) Int. Cl. A61M 29/00

(52) U.S. Cl. 606/192

(57) ABSTRACT

A surgical device is disclosed which is advantageously for use in orthopaedic hand surgery where there exists the requirement to dilate a tendon sheath in order to pass a retrieved or harvested tendon through the dilated sheath. In a disclosed embodiment the device comprises an elongated body having a central bore extending from an open to a closed end, and an inflatable balloon disposed around a portion of the body portion in proximity to the closed end thereof, wherein the balloon is in fluid communication with the central bore via perforations in the outer surface of the body portion. The device also includes an aperture in the closed end for receiving a suture. Thus, the device may be progressively passed through a tendon sheath with the balloon inflated to dilate the sheath, and a tendon portion secured to the aperture in the closed end with a suture so that the tendon portion can be pulled through the dilated sheath by the device.

### FIELD OF INVENTION

The present invention relates to a surgical device, and in particular, but not exclusively, to a surgical device for use in orthopaedics.

### BACKGROUND TO INVENTION

Tendon injuries can be difficult to successfully repair due to the nature in which tendons heal. Tendons are extremely sensitive to injury and physical intervention and are prone to considerable scarring and adhesion formation. Even puncture by a needle and suture or other surgical device can cause significant adhesion formation. The presence of scarring and adhesions can severely affect the movement and gliding of repaired tendons and are often the cause of failure in reconstruction procedures.

Of course, the presence of scarring will be determined by the type of injury and also by the method by which tendons are repaired. It is therefore preferred that relatively atraumatic reconstructive methods be used to minimise tissue reaction and scarring. Furthermore, it is also preferred that surgical intervention be minimised and repair be achieved in a single stage operation.

However, some tendon injuries such as long standing injuries to flexor tendons of the hand currently require a multi-stage operation, or a single stage operation which utilises harsh methods of repair, reducing the likelihood of success.

For example, a long-standing injury involving a severed flexor tendon of the hand will normally result in the tendon retracting from its tendon sheath. In this instance, during tendon grafting or reconstruction procedures the tendon sheath may require dilation which is conventionally achieved by a number of means, such as forcing metal bougies or probes into the sheath, along with other multipurpose instruments to allow sufficient sheath diameter for passage of a tendon graft or artificial material. Occasionally, however, and depending on the type of traumatising injury, the sheath may be too scarred or shrunken to allow this and the sheath has to be incised and refashioned/reattached to allow grafting. These methods normally involve a single stage operation and are relatively traumatic methods which may increase the risk of further scarring and an unsuccessful or non-gliding repair.

If the problem or injury is too severe for the above methods to be readily executed, then a multi stage operation may be required which may involve, for example, incising the sheath and implanting a silicone rod therein and allowing the tissue sufficient time to reform over the rod. Once the sheath has reformed, the tendon grafting procedure can be carried out.

It is an object of at least one aspect of the present invention to obviate or at least mitigate the aforementioned problems in the prior art.

### SUMMARY OF INVENTION

According to a first aspect of the present invention, there is provided a surgical device for use in or when used in orthopaedics, said device being adapted for dilating a tendon sheath.

The device may conveniently be termed a “catheter device”.

The Applicant has conveniently called the device the “TENOCATH” (Trade Mark) device.

According to a second aspect of the present invention, there is provided a surgical device, said device having means for dilating an orifice in a body part, and means for engaging an elongate member.

By said device the elongate member may be pulled through the dilated orifice.

Conveniently, the body part is a tendon sheath.

Conveniently also, the elongate member is a flexible cord-like structure.

The cord-like structure may be a retrieved tendon, a tendon graft or an artificial tendon material or the like.

Thus, the device of the present invention may be used as both a dilator and a passer, particularly adapted for use in reconstruction of tendons, for example, allowing a tendon sheath to be dilated and a tendon stump, tendon graft or suitable artificial material to be pulled therethrough in a relatively atraumatic manner. This, therefore, in a number of cases, allows a complete repair to be achieved in a single stage operation.

The dilating means may comprise a deflatably inflatable balloon, and the engaging means may comprise an eye for suturing the tendon or suitable artificial material to the device.

According to a third aspect of the present invention, there is provided a surgical device comprising:
an elongate body portion having a central bore extending from an open end to a closed end thereof;
means for receiving a syringe, said means coupled to the open end of the body portion;
at least one aperture in the closed end of the body portion, said at least one aperture adapted for receiving a suture; and
an inflatable balloon disposed around a portion of the body portion in proximity to the closed end thereof, said balloon forming a fluid tight seal with the outer surface of the body portion and being in fluid communication with the central bore via at least one perforation disposed on the outer surface of the body portion.

Although the surgical device of the present invention may be used for any purpose involving the requirement to dilate tissue, the device is particularly adapted and advantageous for use in tendon reconstructive surgery, such as in surgery to repair divided flexor or extensor tendons, for example, which have become removed from their synovial sheaths.

Preferably, the surgical device is for use in performing surgery to repair a divided flexor tendon of the hand, such as the tendon of the flexor digitorum profundus or the flexor digitorum superficialis which extend from the forearm to the base of the distal and middle phalanges respectively of the fingers.

More preferably, the surgical device is for use in performing surgery to repair long-standing tendon injuries with significant scarring and narrowing of the sheath system.

Thus, in such an application, a tendon sheath from which a tendon has become removed may be dilated by inflating the balloon while the device is gradually inserted into the sheath. Once the sheath has been dilated to the required extent, the retrieved tendon, tendon graft, artificial tendon material or the like is sutured to the aperture in the closed end of the body portion when the device is inserted through the sheath, and the device is then removed from the sheath, thus pulling the tendon or artificial material therethrough.

Additionally, to ensure that the sheath has been dilated to the required extent, the balloon may be semi-inflated and passed through the tendon sheath, which would allow restrictions to be identified.

The present invention therefore permits relatively atraumatic repair of damaged tendons and the like while minimising scarring of tissue and thus the density of adhesions which can increase the risk of unsuccessful repair and hence poor functional outcome.

Furthermore, the present invention also allows tendon repair to be achieved in a single-stage operation.

Preferably, the balloon is inflated with a sterile fluid injected into the central bore of the body portion and through the at least one perforation using a syringe coupled thereto.

Preferably also, the balloon is deflated by elastic recovery of the balloon material which forces the sterile fluid out of the balloon and into the central bore of the body portion via the at least one perforation disposed on the outer surface thereof.

Conveniently, the elongate body portion of the device is flexible.

Preferably, the body portion is made of a material having a low frictional coefficient to facilitate easy passage through a tendon sheath or the like.

The body portion may be made of a plastics material such as polyvinyl chloride (PVC) or other plastic of medical grade (United States Plastic (USP) grade IV or VI).

In a preferred embodiment, the means for receiving a syringe is a luer lock having an outer diameter greater than that of the body portion and having a throughbore coaxially aligned with the central bore of the body portion.

Advantageously, the luer lock comprises flange portions allowing the luer lock to be gripped by the syringe while the syringe is inserted and used therein. The syringe may be a conventional syringe, or alternatively, may comprise a threaded collar which is adapted to engage a corresponding threaded portion on the luer lock such that the syringe may be coupled thereto.

Preferably, the balloon is manufactured from latex, or alternatively from any other suitable resilient material including silicone rubber.

Conveniently, the balloon may have a fluid capacity of between 0.05 ml to 1.1 ml.

Preferably, the balloon has a wall thickness of approximately 0.3 mm thick.

Preferably, the balloon is detachable from the body portion at the side nearest the open end thereof. This feature allows the balloon to be drawn over the aperture in the closed end to provide a smooth interface between the device and a tendon graft or the like which may be sutured to the aperture in the closed end of the device. Thus, in this capacity, the balloon may act as an end sheath for the tendon graft, for example.

Advantageously, the outer diameter of the portion of the body around which the balloon is disposed may be reduced such that the body portion has a substantially constant outer diameter when the balloon is deflated. This allows for smooth passage of the device through a tendon sheath, for example, and prevents damage of the balloon when the device is not in use.

In a preferred embodiment of the present invention, the body portion is tapered from the open end to the closed end thereof. This allows the device to be more easily inserted into a tendon sheath, for example.

Preferably, the device is sterile and is provided in a hermetic packaging to prevent contamination until such time as the device is required.

Preferably, the device has a length of approximately 0.5 m or less. More preferably, the device has a length of approximately 0.25 m.

Preferably, the body portion has an outer diameter of approximately 2 mm.

The body portion may have an inner diameter of approximately 1 mm or less and preferably between 0.5 mm to 1 mm.

Preferably, the portion of the body portion around which the balloon is disposed has an outer diameter of approximately 1.6 mm.

Preferably also, the length of the body portion around which the balloon is disposed is approximately 10 mm for a balloon capacity of approximately 1 ml.

Preferably, the luer lock has a length of approximately 20 mm and an outer diameter ranging from 6 mm at an open end to 5 mm at the end coupled to the body portion. Preferably also, the luer lock throughbore has an open diameter of approximately 4.4 mm. Additionally, preferably the flange portion of the luer lock has an outer diameter of approximately 7.5 mm.

Preferably, the aperture provided in the closed end of the device has a diameter of approximately 0.7 mm.

According to a fourth aspect of the present invention, there is provided a method of performing surgery, said method comprising the steps of:
(1) providing a surgical device having means for dilating an orifice in a body part, and means for engaging an elongate structure;
(2) inserting said device into the orifice in the body part and activating said dilating means to dilate said orifice;
(3) engaging the elongate member and retracting the device from the orifice to pull the elongate member therethrough.

According to a fifth aspect of the present invention, there is provided a method of performing surgery, said method comprising the steps of:
(1) providing a surgical device comprising: an elongate body portion having a central bore extending from an open end to a closed end thereof; means for receiving a syringe, said means coupled to the open end of the body portion; at least one aperture in the closed end of the body portion, said at least one aperture adapted for receiving a suture; and an inflatable balloon disposed around a portion of the body portion in proximity to the closed end thereof, said balloon forming a fluid tight seal with the outer surface of the body portion and being in fluid communication with the central bore via at least one perforation disposed on the outer surface of the body portion;
(2) inserting the device into a tendon sheath;
(3) inflating the balloon to its maximum capacity with a sterile fluid and retaining said balloon in an inflated state for a discrete time period;
(4) deflating the balloon and retracting the device incrementally and repeating step (3) until the entire length of the sheath has been dilated;
(5) suturing a cord-like structure to the aperture in the closed end portion of the elongate body portion; and
(6) retracting the device from the tendon sheath to pull the cord-like structure therethrough.

The method may comprise a method of repairing a tendon. In this regard, the applicant has conveniently termed the method “SHEATHPLASTY” and “BALLOON FLEXOR SHEATHPLASTY”.

Preferably, the method further comprises the step of semi-inflating the balloon and passing the catheter through the sheath to ensure proper dilation.

### BRIEF DESCRIPTION OF DRAWINGS

These and other aspects of the present invention will now be described, by way of example only, with reference to the accompanying drawings in which:

FIG. 1 is a side view of a surgical device in accordance with a preferred embodiment of the present invention;

FIG. 2 is an enlarged view of an end portion of the device of FIG. 1;

FIG. 3 is an end view of another end of the device of FIG. 1;

FIGS. 4 and 5 show the device of FIG. 1 in use; and

FIGS. 6 and 7 show the device of FIG. 1 in use in a surgical procedure to repair a divided tendon of the hand.

### DETAILED DESCRIPTION OF DRAWINGS

While it would be clear to a person of skill in the art that the surgical device of the present invention may be used in a number of procedures, in the interests of brevity and clarity, the following description of prefered embodiments relates to a surgical device particularly advantageously for use in tendon surgery.

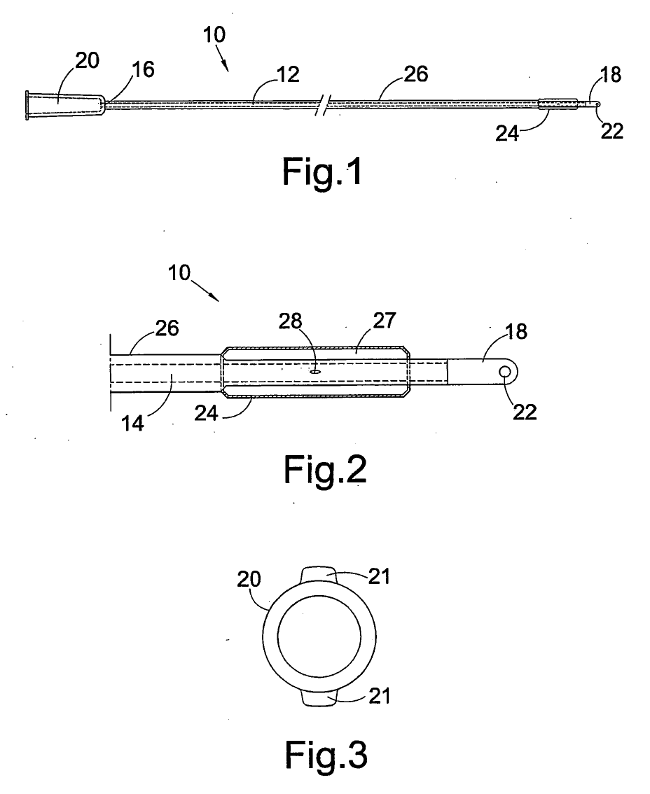

Reference is first made to FIGS. 1 and 2 of the drawings in which there is shown a surgical device 10 in accordance with a preferred embodiment of the present invention, wherein FIG. 2 is an enlarged view of an end portion of the device shown in FIG. 1.

The device 10 comprises a plastic flexible elongate body portion 12 which has a central bore 14 (FIG. 2) which extends from an open end 16 to a closed end 18 of the body portion 12. Coupled to the open end 16 is a luer lock 20 for recieving a syringe (not shown). The luer lock 20 has an outer diameter greater than that of the body portion 12 and has a throughbore (not shown) coaxially aligned with the central bore 14 of the body portion 12. Additionally, the luer lock 20 comprises flange portions 21, shown in FIG. 3, allowing the luer lock to be gripped by the collar of a syringe while a syringe is inserted and used therein.

An aperture 22 is provided in the closed end 18 of the body portion 12, wherein the aperture 22 is adapted for receiving a suture to attach a tendon or other suitable artificial material to the device 10.

Disposed around a portion 27 of the body portion 12 in proximity to the closed end 18 thereof is a latex balloon 24 which forms a fluid tight seal with the outer surface 26 of the body portion 12 and is in fluid communication with the central bore 14 via perforations 28 (only one shown in FIG. 2) disposed on the outer surface 26 of the body portion 12.

As clearly shown in FIG. 2, the outer diameter of the portion of the body 12 around which the balloon 24 is disposed is reduced such that the body portion 12 has a substantially constant outer diameter when deflated. This allows the device to be easily passed through tendon sheaths, for example.

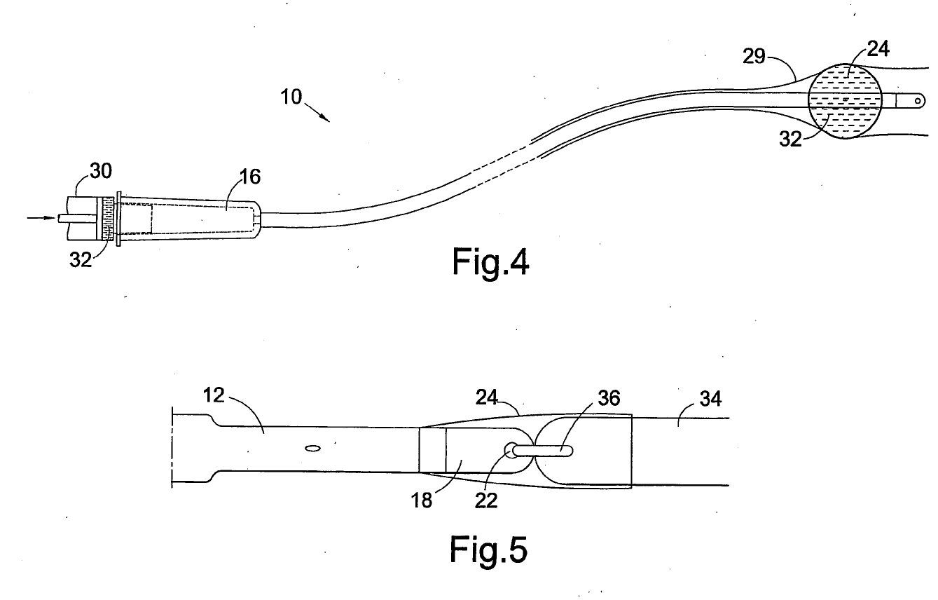

Reference is now made to FIG. 4 in which there is shown the device 10 of FIG. 1 in use. The device 10 is located within a tendon sheath 29, and a syringe 30 containing a sterile fluid 32 such as saline solution is coupled to the luer lock 16. The sterile fluid 32 is injected into the device through the central bore 14 (not shown) and into the balloon 24 causing the balloon to inflate in order to dilate the tendon sheath 29. To dilate the entire length of the sheath 29, the device 10 is progressively retracted and the balloon 24 reinflated.

Once the entire sheath has been dilated to the required extent, the device is passed therethrough and a tendon graft 34, for example, is fixed to the aperture 22 with a suture 36, as shown in FIG. 5. One end of the balloon 24 is detached from the body portion 12 and is drawn over the aperture 22 in the closed end 18 to provide a smooth interface between the device 10 and the tendon graft 34. This protects the tendon graft 34 and prevents it from becoming detached from the device 10 when the device is retracted from the sheath in order to pull the tendon graft 34 therethrough.

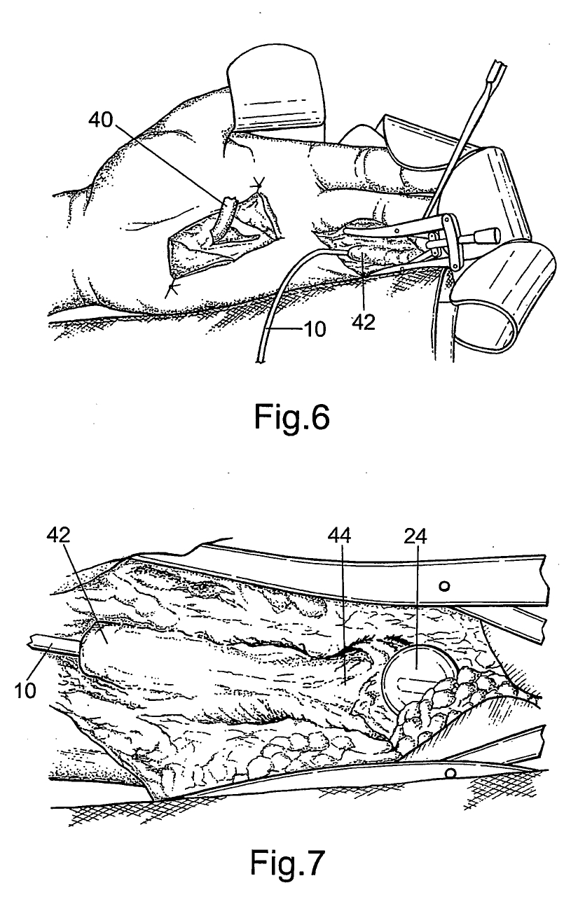

Reference is now made to FIGS. 6 and 7 of the drawings in which there is shown the device 10 of the present invention in use in a surgical procedure to repair a divided flexor digitorum profundus tendon of the hand. In this particular case, the tendon has become detached from the distal phalanx of the ring finger and the tendon stump 40 has become retracted from its synovial sheath 42, resulting in contraction of the sheath 42.

The sheath 42 has been exposed and the device 10 has been inserted therein from proximally to distally, that is, from the end nearest the palmar surface of the hand to the top of the finger. As shown in FIG. 7, the device 10 is fully inserted to reveal the balloon 24, which has been inflated for clarity. The operative technique then involves aligning the balloon with the distal end 44 of the sheath 42 and inflating to its maximum capacity of approximately 1 ml, and retaining the balloon 24 in this state for approximately 1 minute to allow the tissue to equilibrate. The balloon 24 is then deflated and the device retracted by one or two centimetres, and the process repeated until the entire length of the sheath 42 has been sufficiently dilated. Once completed, the balloon 24 may be semi-inflated and passed through the sheath 42 to check adequate dilation.

The stump of the retracted tendon 40 is then isolated and palmaris longus tendon, for example, is harvested to be used as a tendon graft. The distal end of the graft is then anchored to the base of the distal phalanx using a pullout suture technique over a dental roll, for example. The graft is then sutured to the aperture 22 in the device which is proximally pulled through the dilated sheath 42. The proximal end of the tendon graft is sutured to the profundus stump using an interlacing technique, for example.

Thus, using the device 10 of the present invention in the manner discussed above allows a relatively atraumatic,

It would be apparent to a person of skill in the art that the embodiments hereinbefore described are merely exemplary of the present invention and various modifications may be made thereto without departing from the scope of the invention. For example, the device is particularly beneficial in but not limited for use in orthopaedics. The device is most advantageously used in tendon surgery, however the novel devices of the second and third aspects may be used in other surgical fields where the requirement for the dilation of tissue exists. Further, the device may be used in tendon harvesting procedures. The body portion may be manufactured from any suitable flexible material. Additionally, the balloon may be made of any suitable resilient material. The dimensions may be selected in accordance with the required use of the device.

1. A surgical device comprising:
an elongate body portion having a central bore extending from an open end to a closed end thereof;
means for receiving a syringe, said means coupled to the open end of the body portion;
at least one aperture in the closed end of the body portion, said at least one aperture adapted for receiving a suture; and
an inflatable balloon disposed around a portion of the body portion in proximity to the closed end thereof, said balloon forming a fluid tight seal with the outer surface of the body portion and being in fluid communication with the central bore via at least one perforation disposed on the outer surface of the body portion, wherein the balloon is detachable from the body portion at the side nearest the open end thereof.

2. A surgical device as claimed in claim 1, adapted for use in tendon reconstructive surgery.

3. A surgical device as claimed in claim 1, wherein the surgical device is for use in performing surgery to repair a divided flexor tendon of the hand.

4. A surgical device as claimed in claim 1, wherein, the surgical device is for use in performing surgery to repair long-standing tendon injuries with significant scarring and narrowing of the sheath system.

5. A surgical device as claimed in claim 1, wherein the surgical device is for use in performing surgery to repair tendon injuries in a single-stage operation.

6. A surgical device as claimed in claim 1, wherein the balloon is inflatable with a sterile fluid injected into the central bore of the body portion and through the at least one perforation using a syringe coupled thereto.

7. A surgical device as claimed in claim 6, wherein the balloon is deflatable by elastic recovery of the balloon material which forces the sterile fluid out of the balloon and into the central bore of the body portion via the at least one perforation disposed on the outer surface thereof.

8. A surgical device as claimed in any one of claim 1, wherein the elongate body portion of the device is flexible.

9. A surgical device as claimed in any one of claim 1, wherein the body portion is made of a material having a low frictional coefficient to facilitate easy passage through a tendon sheath.

10. A surgical device as claimed in any one of claim 1, wherein the body portion is made of a plastics material.

11. A surgical device as claimed in claim 1, wherein the body portion is made of polyvinyl chloride (PVC).

12. A surgical device as claimed in claim 1, wherein the body portion is made of a plastic of medical grade, being United States Plastic (USP) grade IV or VI.

13. A surgical device as claimed in claim 1, wherein the means for receiving a syringe is a luer lock having an outer diameter greater than that of the body portion and having a throughbore coaxially aligned with the central bore of the body portion.

14. A surgical device as claimed in claim 13, wherein the luer lock comprises flange portions allowing the luer lock to be gripped by the syringe while the syringe is inserted and used therein.

15. A surgical device as claimed in claim 1, wherein the means for receiving a syringe comprises a threaded portion for engaging a threaded collar mounted on a syringe.

16. A surgical device as claimed in claim 1, wherein the balloon is manufactured from a resilient material.

17. A surgical device as claimed in claim 16, wherein the balloon is manufactured from latex.

18. A surgical device as claimed in claim 1, wherein the balloon has a fluid capacity of between 0.05 ml to 1.1 ml.

19. A surgical device as claimed in claim 1, wherein the balloon has a wall thickness of approximately 0.3 mm.

20. A surgical device as claimed in claim 1, wherein the outer diameter of the portion of the body around which the balloon is disposed is reduced such that the body portion has a substantially constant outer diameter when the balloon is deflated.

21. A surgical device as claimed in claim 1, wherein the body portion is tapered from the open end to the closed end thereof.

22. A surgical device as claimed in claim 1, wherein the device is sterile and is provided in a hermetic packaging to prevent contamination until such time as the device is required.

23. A surgical device as claimed in claim 1, wherein the device has a length of approximately 0.5 m or less.

24. A surgical device as claimed in claim 1, wherein the device has a length of approximately 0.25 m.

25. A surgical device as claimed in claim 1, wherein the body portion has an outer diameter of approximately 2 mm.

26. A surgical device as claimed in claim 1, wherein the body portion has an inner diameter of approximately 1 mm or less.

27. A surgical device as claimed in claim 1, wherein the body portion has an inner diameter of between 0.5 mm to 1 mm.

28. A surgical device as claimed in claim 1, wherein the portion of the body portion around which the balloon is disposed has an outer diameter of approximately 1.6 mm.

29. A surgical device as claimed in claim 1, wherein the length of the body portion around which the balloon is disposed is approximately 10 mm for a balloon capacity of approximately 1 ml.

30. A surgical device as claimed in claim 13, wherein the luer lock has a length of approximately 20 mm and an outer diameter ranging from 6 mm at an open end to 5 mm at the end coupled to the body portion.

31. A surgical device as claimed in claim 13, wherein the luer lock throughbore has an open diameter of approximately 4.4 mm.

32. A surgical device as claimed in claim 13, wherein the flange portion of the luer lock has an outer diameter of approximately 7.5 mm.

33. A surgical device as claimed in claim 1, wherein the aperture provided in the closed end of the device has a diameter of approximately 0.7 mm.

34. A surgical device having means for dilating an orifice in a body part and means for engaging an elongate member, wherein the dilating means comprises a deflatably inflatable balloon mounted on a body portion, the engaging means comprising an aperture on the body portion, and the balloon being partially detachable from the body portion so as to be capable of being drawn over the aperture.

35. A surgical device as claimed in claim 34 adapted for pulling said elongate member through the dilated orifice.

36. A surgical device as claimed in claim 34, wherein the body part is a tendon sheath.

37. A surgical device as claimed in claim 34, wherein the elongate member is a flexible cord-like structure.

38. A surgical device as claimed in claim 37, wherein the cord-like structure is a retrieved tendon.

39. A surgical device as claimed in claim 37, wherein the cord-like structure is a tendon graft.

40. A surgical device as claimed in claim 37, wherein the cord-like structure is an artificial tendon material.

41. A surgical device as claimed in claim 34, wherein the surgical device is adapted for use as both a dilator and a passer.

42. A surgical device as claimed in claim 34, wherein the surgical device is adapted for use in reconstruction of tendons.

43. A surgical device as claimed in claim 34, wherein the balloon is mounted on the body portion at two points of attachment, the balloon being detachable from the body portion at an attachment point furthest removed from the aperture.

44. A method of performing surgery, said method comprising the steps of:
(1) providing a surgical device having means for dilating an orifice in a body part, and means for engaging an elongate structure;
(2) inserting said device into the orifice in the body part and activating said dilating means to dilate said orifice;
(3) engaging the elongate member and retracting the device from the orifice to pull the elongate member therethrough.

45. A method of performing surgery, said method comprising the steps of:
(1) providing a surgical device comprising: an elongate body portion having a central bore extending from an open end to a closed end thereof; means for receiving a syringe, said means coupled to the open end of the body portion; at least one aperture in the closed end of the body portion, said at least one aperture adapted for receiving a suture; and an inflatable balloon disposed around a portion of the body portion in proximity to the closed end thereof, said balloon forming a fluid tight seal with the outer surface of the body portion and being in fluid communication with the central bore via at least one perforation disposed on the outer surface of the body portion;
(2) inserting the device into a tendon sheath;
(3) inflating the balloon to its maximum capacity with a sterile fluid and retaining said balloon in an inflated state for a discrete time period;
(4) deflating the balloon and retracting the device incrementally and repeating step (3) until the entire length of the sheath has been dilated;
(5) suturing a cord-like structure to the aperture in the closed end portion of the elongate body portion; and
(6) retracting the device from the tendon sheath to pull the cord-like structure therethrough.

46. A method of performing surgery as claimed in claim 45 comprising a method of repairing a tendon.

47. A method of performing surgery as claimed in claim 45, wherein the method further comprises the step of semi-inflating the balloon and passing the catheter through the sheath to ensure proper dilation.

48. A surgical device for use in or when used in orthopaedics, said device being adapted for dilating a tendon sheath.
